(19) United States
(12) Patent Application Publication
(10) Pub. No.: US 2024/0226300 A1
(43) Pub. Date: Jul. 11, 2024

(54) TREATMENT OF EXPOSURE TO VESICANTS

(71) Applicant: Judith Boston, Manhattan Beach, CA (US)

(72) Inventor: Judith Boston, Manhattan Beach, CA (US)

(21) Appl. No.: 18/416,349

(22) Filed: Jan. 18, 2024

Related U.S. Application Data
(63) Continuation of application No. PCT/US2022/037634, filed on Jul. 19, 2022.
(60) Provisional application No. 63/223,421, filed on Jul. 19, 2021.
(60) Provisional application No. 63/320,625, filed on Mar. 16, 2022.
(60) Provisional application No. 63/349,253, filed on Jun. 6, 2022.

(51) Int. Cl. A61K 45/00; A61P 7/00

(52) U.S. Cl. CPC A61K 45/00; A61P 7/00

(57) ABSTRACT

This disclosure relates to the use of an oxygen-containing liquid as a counter measure against vesicants.

### CROSS-REFERENCE TO RELATED APPLICATIONS

This application is a continuation of International Pat. App. No. PCT/US2022/037634, filed Jul. 19, 2022, which claims the benefit of U.S. Provisional Patent Application No. 63/223,421, filed Jul. 19, 2021, U.S. Provisional Patent Application No. 63/320,625, filed Mar. 16, 2022, and U.S. Provisional Patent Application No. 63/349,253, filed Jun. 6, 2022, all of which are incorporated by reference herein in their entirety.

### SUMMARY

This disclosure relates to the use of an oxygen-containing liquid as a counter measure against vesicants, or for treating exposure to vesicants.

This disclosure relates to the use of an oxygen-containing liquid for treating conditions related to ischemia, hypoxia, an alteration in electrochemistry, VEGF, HIF, or a reactive oxygen species, cancer, and other conditions.

This disclosure relates to the use of an oxygen-containing liquid for cytoprotection, or for protection against pathogen and/or hypoxia induced cell damage.

This disclosure also relates to the use of an oxygen-containing liquid to oxygenate blood, such as blood in a human being, without a ventilator.

Some embodiments include a method of treating an ocular condition comprising administering or delivering an oxygen-containing liquid to the eye of a mammal suffering from an ocular condition.

### BRIEF DESCRIPTION OF FIGURES

FIG. 1 depicts the scotopic b-wave response of ischemic rabbit eyes subjected to treatment with a hyperbaric oxygen solution as compared to controls.

FIG. 2 depicts the levels of VEGF in retinal pigment epithelium (RPE) cells exposed to hypoxic conditions and treated with a hyperbaric oxygen solution.

FIG. 3 depicts the levels of HIF in RPE cells exposed to hypoxic conditions and treated with a hyperbaric oxygen solution.

FIG. 4 depicts two possible embodiments of nanoparticles containing an oxygen-containing liquid.

FIG. 5 depicts an embodiment of nanoparticles containing an oxygen-containing liquid and further dispersed within an oxygen-containing liquid.

### DETAILED DESCRIPTION

In some embodiments, an oxygen-containing liquid further contains an immune system booster.

Reference to “oxygen-containing liquid” herein includes administration of an actual oxygen containing liquid, or another form that can generate oxygen in a liquid in a human or animal body, including the compositions, forms, delivery systems, etc. described herein.

For treatment of any condition described herein, the oxygen-containing liquid may contain chromium, such as a chromium complex, chromium-containing compound, a chromium-containing ion, or a chromium ion. Alternatively, chromium, such as a chromium complex, chromium-containing compound, a chromium-containing ion, or a chromium ion may be used in a solid or liquid dosage form, such as an aqueous liquid or dispersion, for the treatment of any condition described herein.

In some embodiments, an oxygen-containing liquid may be used with diseased animal or human cells, such as cancerous human cells, for the diagnosis, study, or treatment of infections.

In some embodiments, the oxygen-containing liquid may be delivered by a pump, such as a pump similar to a pump used to deliver insulin.

In some embodiments, an oxygen-containing liquid may be use as a counter measure against vesicants, e.g., distilled mustard, mustard gas, sulfur mustard, nitrogen mustard, sesqui mustard, Lewisite, ammonium oxide, sulfur oxide, phosgene oxime, etc.

In some embodiments, an oxygen-containing liquid may be used to prevent or reduce the likelihood of being infected after exposure, or to prevent or reduce the severity of symptoms after exposure.

In some embodiments, an oxygen containing liquid may be used with a diagnostic. For example, an oxygen containing liquid can be tagged, e.g. with a fluorescent tag, to detect early hypoxic areas that may be predisposed to developing a disease such as cancer.

In some embodiments, an oxygen containing liquid, including an aqueous oxygen containing liquid and/or an oxygen containing hydrogel, may be administered in conjunction with corneal collagen cross-linking, including corneal collagen cross-linking with riboflavin and/or UV-A. In some embodiments, patients who have corneal collagen cross-linking are treated with an oxygen containing liquid for at least 1 day, at least 1 week, or at least 2 weeks after the procedure is performed.

In some embodiments, an oxygen-containing liquid may be used to modulate VEGF. In some embodiments, an oxygen-containing liquid may be used to modulate inflammatory mediators.

In some embodiments, an oxygen containing liquid is delivered via dropless methods of delivery, such as intracameral, through the punctal delivery, or intravitreal, hydrogel plugs, implants, or subtenon injection.

In some embodiments, the oxygen containing liquid is in a hydrogel.

In some embodiments, an oxygen-containing liquid is used to improve healing of cells and/or adjust/normalize vascular and cellular function. In some embodiments, an oxygen-containing liquid is used to stop or slow blood vessel leakage. In some embodiments, a nanoparticle containing an oxygen-containing liquid is used for treatment of cancer or an ischemic eye condition.

In some embodiments, an oxygen-containing liquid may be included in particles, such as nanoparticles or microparticles. In some embodiments, an oxygen-containing liquid may be contained within a drug delivery device. In some embodiments, a device that releases an oxygen-containing liquid may be constructed from a nano particle or other structure containing an oxygen-containing liquid. In some embodiments, such a device may be prepared using a 3D printer, which may be used to make a scaffold or oxygenated micro fibers.

This disclosure relates to methods of treating ischemic conditions, such as ocular ischemic conditions, other conditions related to hypoxia, or conditions related to reactive oxygen species, comprising administering or delivering an oxygen-containing liquid to a mammal, such as a human being, for the treatment of the condition. The oxygen-containing liquid may be used alone or in combination with other drugs or therapeutic agents, such as a hypoxia-inducible factor 1 (HIF-1) inhibitor (e.g. a eudistidine).

The term “treating” or “treatment” broadly includes any kind of treatment activity, including the diagnosis, cure, mitigation, or prevention of disease in man or other animals, or any activity that otherwise affects the structure or any function of the body of man or other animals.

The oxygen-containing liquid may be any liquid composition containing oxygen, or a compound that provides an oxygen pressure to a liquid, or a liquid which provides oxygen to mammalian cells or tissue, which is suitable for use in a mammal, including a human being, for therapeutic purposes. The oxygen-containing liquid may be aqueous, or may be based upon a suitable organic solvent, or may be a combination of aqueous and organic solvents. The liquid may be in the form of a solution, or a multiple phase liquid, such as a suspension, a colloid, an emulsion, a shear-thinning gel, etc. For many routes of administration, such as injections, it may be important for the oxygen-containing liquid to be sterile.

An oxygen-containing liquid may be formulated for any desirable route of delivery including, but not limited to, parenteral, suppository, intravenous, intradermal (e.g. intradermal injection), subcutaneous, oral, inhalative, metered dose inhaler (MDI), transdermal, patch, drops, topical to an eye (e.g. eye drops for delivery to the anterior segment of the eye or eyedrops for delivery to the posterior segment of the eye) or to skin, transmucosal, rectal, intravaginal, intraperitoneal, intramuscular, intralesional, intranasal, subcutaneous (e.g. subcutaneous injection), buccal, intraocular injection, intravitreal injection, sub-retinal injection, intrathecal injection (e.g. directly into the heart), etc. The term “injection” includes injection of a pharmaceutical composition, insertion of an implant or drug delivery device, as well as other types of injections.

In some embodiments, rather than being directly administered, an oxygen-containing liquid may be generated in the target tissue by inserting an implant or drug delivery device into or near the target tissue, which could provide long term delivery of the oxygen-containing liquid. For example, the implant could comprise a biodegradable or bioerodible polymer having components of an oxygenating composition dispersed in the polymer. As the polymer degrades or erodes, the components of the oxygenating composition will mix in the aqueous environment of the tissue into which the implant is inserted, thus generating an oxygen-containing liquid at or near the tissue to be targeted. The implant or device may be administered by any route described above, including intravenously (e.g. by injection), intravitreally (e.g. by injection), or subretinally (e.g. by injection). Oxygen-containing liquid may also be generated by other types of solid devices, such as punctal plugs and contact lenses containing components of the oxygenating composition, which gradually diffuse out of the devices. Alternatively, a punctal plug or contact lens might be biodegradable or bioerodible.

Any therapeutic composition, dosage form, drug delivery device, implant, etc. described herein (e.g. by oral, IV, etc.) may be formulated or designed for timed release, delayed release, controlled release, sustained release, etc., e.g. by the use of biodegradable polymers or bioerodible materials. A composition, dosage form, drug delivery device, implant, etc., may provide targeted delivery by both the route and/or location of administration and by including materials or structural features that are designed to respond to the specific environment, chemical properties, chemical activity, biological properties, or biological activity of a type of cell, tissue, organ, or biological system to be targeted.

Examples of materials with timed release, delayed release, controlled release, sustained release, etc. properties include silica-based materials, or organic biodegradable materials, such as polymers comprising poly (D,L-lactic acid) (PLA) and poly (D,L-lactic-co-glycolic acid) or poly (lactic-co-glycolic acid) (PLGA), polyesteramide (PEA, DSM chemical), and polycaprolactone (PCL); hydrogels, such as polyvinyl alcohols (PVA), PEG amines, PEG-N-hydroxysuccinamide esters and the like; collagen based materials; acrylic acid and methacrylic acid copolymers and various esters thereof, e.g. methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polyacrylamide, poly(methacrylic acid anhydride), and glycidyl methacrylate copolymers; polymerizable quaternary ammonium compounds, e.g. quaternized aminoalkyl esters and aminoalkyl amides of acrylic acid and methacrylic acid, for example β-methacryloxyethyltrimethylammonium methosulfate, β-acryloxypropyltrimethylammonium chloride, and trimethylaminomethylmethacrylamide methosulfate, etc. The quaternary ammonium atom can also be part of a heterocycle, as in methacryloxyethylmethylmorpholinium chloride or the corresponding piperidinium salt, or it can be joined to an acrylic acid group or a methacrylic acid group by way of a group containing hetero atoms, such as a polyglycol ether group. Further suitable polymerizable quaternary ammonium compounds include quaternized vinyl-substituted nitrogen heterocycles such as methyl-vinyl pyridinium salts, vinyl esters of quaternized amino carboxylic acids, styryltrialkyl ammonium salts, and the like. Other polymerizable quaternary ammonium compounds include benzyldimethylammoniumethylmethacrylate chloride, diethylmethylammoniumethyl-acrylate and -methacrylate methosulfate, N-trimethylammoniumpropylmethacrylamide chloride, and N-trimethylammonium-2,2-dimethylpropyl-1-methacrylate chloride.

In some embodiments, a delivery system, such as a microparticle or nanoparticle delivery system (e.g. PLGA nanoparticles), may provide extended delivery of the oxygen containing liquid to a patient. For example, the delivery system may provide oxygen to the target cells, tissue, organ, or system (e.g. IV nanoparticles, such as PLGA nanoparticles, containing the oxygen containing liquid, may provide oxygen to the blood) over an extended period of time, such as at least about 1 hour, at least about 4 hours, at least about 8 hours, at least about 12 hours, at least about 24 hours, at least about 2 days, at least about 3 days, at least about 4 days, at least about 5 days, at least about 6 days, at least about 7 days, at least 2 weeks, at least 3 weeks, at least 4 weeks, at least 6 weeks, at least 2 months, at least 3 months, at least 4 months, at least 6 months, about 1-2 days, about 2-4 days, about 4-7 days, about 1-2 weeks, about 2-4 weeks, about 1-2 months, about 2-4 months, about 4-6 months, or about 1-4 months. In some embodiments, administration of the delivery system may be repeated at an interval of about 4-8 hours, about 8-16 hours, about 16-24 hours, about 1-2 days, about 2-4 days, about 4-7 days, about 1-2 weeks, about 2-4 weeks, about 1-2 months, about 2-4 months, about 4-6 months, or about 1-4 months.

PLGA nanoparticles or microparticles may be administered, e.g. by injection, insertion or other means, to treat infection even without an oxygen containing liquid.

In some embodiments, the delivery system is sterile.

In some embodiments, an oxygen containing liquid, or a device or implant that releases or generates the oxygen containing liquid in the body, is directly administered, e.g. by injection, insertion or other means, into a tissue, an organ or body system affected by a condition to be treated, such as an organ or system infected by a virus or bacteria, such as the central nervous system such as, the brain, eyes, nerves; the cardiopulmonary system, such as the heart, lungs, etc.; the liver; the kidneys; musculoskeletal system, the muscles; the endocrine system, such as the pancreas; the gastrointestinal system, such as the esophagus, the stomach, the small intestine, the large intestine, etc.

The oxygen-containing liquid may have a higher partial oxygen pressure than plain water, or a higher partial oxygen pressure than air at atmospheric pressure, for example, at room temperature (e.g. 23° C.) or body temperature (e.g. 37° C.), the oxygen-containing liquid may have an oxygen pressure that is at least 120 mmHg, at least 140 mmHg, at least 145 mmHg, at least 150 mmHg, at least 155 mmHg, at least 160 mmHg, at least 165 mmHg, at least 170 mmHg, up to 180 mmHg, up to 200 mmHg, up to about 250 mmHg, up to about 300 mmHg, up to about 350 mmHg, up to about 400 mmHg, up to about 450 mmHg, up to about 500 mmHg, about 120-500 mmHg, about 20-40 mmHg, about 40-60 mmHg, about 60-80 mmHg, about 80-100 mmHg, about 100-120 mmHg, about 120-140 mmHg, about 140-145 mmHg, about 145-150 mmHg, about 150-155 mmHg, about 155-160 mmHg, about 160-165 mmHg, about 165-170 mmHg, about 170-175 mmHg, about 175-180 mmHg, about 140-150 mmHg, about 150-160 mmHg, about 160-170 mmHg, about 170-180 mmHg, about 180-190 mmHg, about 190-200 mmHg, about 200-210 mmHg, about 210-220 mmHg, about 220-230 mmHg, about 230-240 mmHg, about 240-250 mmHg, about 250-260 mmHg, about 260-270 mmHg, about 270-280 mmHg, about 280-290 mmHg, about 290-300 mmHg, about 300-320 mmHg, about 320-340 mmHg, about 340-360 mmHg, about 360-380 mmHg, about 380-400 mmHg, about 400-420 mmHg, about 420-440 mmHg, about 440-460 mmHg, about 460-480 mmHg, about 480-500 mmHg, about 140-160 mmHg, about 160-180 mmHg, about 180-200 mmHg, about 160-200 mmHg, about 200-250 mmHg, about 250-300 mmHg, about 300-350 mmHg, about 350-400 mmHg, about 400-450 mmHg, about 450-500 mmHg, about 140-200 mmHg, about 200-300 mmHg, about 300-400 mmHg, about 400-500 mmHg, 500-750 mmHg, 750-1,000 mmHg, 1,000-1,250 mmHg, 1,250-1,500 mmHg, about 175 mmHg, or any oxygen pressure in a range bounded by any of these values. In some embodiments, the oxygen-containing liquid is a hyperbaric oxygen solution (e.g. Examples 1-3 below).

While there may be many ways to add oxygen to a liquid, some oxygen-containing liquids may contain an oxygenating composition, such as a compound, or a combination of compounds, that release an oxygen gas, e.g. by a chemical reaction or chemical degradation. Suitable oxygenating compositions may contain metal oxides (such as CaO, MgO, etc.), metal hydroxides (such as Ca(OH)₂, Mg(OH)₂), peroxides (such as hydrogen peroxide or an organic peroxide), or combinations thereof. Other ingredients may be added to increase or reduce the rate of oxygen release, depending upon the particular need. For example, faster oxygen release may provide higher oxygen pressure. On the other hand, slower oxygen release may provide a longer, more consistent, or more sustained, oxygen pressure. Examples of suitable oxygenating compositions are described in U.S. Pat. No. 8,802,049, which is incorporated by reference herein in its entirety. One useful oxygenating composition contains about 20-30% Ca(OH)₂, about 10-15% H₂O₂, about 0.5-5% sodium acetate, about 0.5-5% KH₂PO₄, and about 1-20% Carrageenan, based upon the total weight of the oxygen-containing liquid. In some embodiments, the total amount of oxygen atoms present in all metal oxides, metal hydroxides, and peroxides present in the oxygen-containing liquid is about 20-70%, about 20-50%, about 50-70%, about 20-30%, about 30-40%, about 40-50%, about 50-60%, about 60-70%, about 70-90%, or about 80-95% of the total weight of the oxygen-containing liquid.

As mentioned above, the components of these oxygenating compositions, such as metal oxides, metal hydroxides, and/or peroxides, may be dispersed in a bioerodible or biodegradable polymer, such as a silicon-based polymer, a polyester, a polyorthoester, a polyphosphoester, a polycarbonate, a polyanhydride, a polyphosphazene, a polyoxalate, a poly(amino acid), a polyhydroxyalkanoate, a polyethyleneglycol, a polyvinylacetate, a polyhydroxyacid, a polyanhydride, or copolymer or blend thereof (e.g. a co-polymer of lactic and glycolic acid).

Components of oxygen-containing liquids or oxygenating compositions, such as metal oxides, metal hydroxides, and/or peroxides, may be lyophilized for oral delivery (e.g. in a tablet, capsule, or other solid oral dosage form), reconstitution, or dispersion into a solid or multiphase (e.g. nanoparticles, nanoemulsions, etc.) drug delivery system or device, such as any delivery system or device described herein. Oral dosage forms comprising an oxygen-containing liquid, or a lyophilized composition, or other composition that can generate an oxygen-containing liquid in a human or animal body, may have an enteric coating.

Appropriate excipients for use in an oxygen-containing liquid may include, for example, one or more carriers, binders, fillers, vehicles, tonicity agents, buffers, disintegrants, surfactants, dispersion or suspension aids, thickening or emulsifying agents, preservatives, lubricants and the like or combinations thereof, as suited to a particular dosage from desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. This document is incorporated herein by reference in its entirety.

In addition to solvent, oxygen, and/or oxygenating compositions, a liquid dosage form for IV, injection (e.g. intraocular injection, sub-retinal injection, intrathecal, directly into the heart), topical (e.g. to an eye), or oral administration to a mammal, including a human being, may contain excipients such as bulking agents (such as mannitol, lactose, sucrose, trehalose, sorbitol, glucose, raffinose, glycine, histidine, polyvinylpyrrolidone, etc.), tonicity agents (e.g. dextrose, glycerin, mannitol, sodium chloride, etc.), buffers (e.g. acetate, e.g. sodium acetate, acetic acid, ammonium acetate, ammonium sulfate, ammonium hydroxide, citrate, tartrate, phosphate, triethanolamine, arginine, aspartate, benzenesulfonic acid, benzoate, bicarbonate, borate, carbonate, succinate, sulfate, tartrate, tromethamine, diethanolamine etc.), preservatives (e.g. phenol, m-cresol, a paraben, such as methylparaben, propylparaben, butylparaben, myristyl gamma-picolinium chloride, benzalkonium chloride, benzethonium chloride, benzyl alcohol, 2-phenoxyethanol, chlorobutanol, thimerosal, phenylmercuric salts, etc.), surfactants (e.g. polyoxyethylene sorbitan monooleate or Tween 80, sorbitan monooleate polyoxyethylene sorbitan monolaurate or Tween 20, lecithin, a polyoxyethylene-polyoxypropylene copolymer, etc.), solvents (e.g. propylene glycol, glycerin, ethanol, polyethylene glycol, sorbitol, dimethylacetamide, Cremophor EL, benzyl benzoate, castor oil, cottonseed oil, N-methyl-2-pyrrolidone, PEG, PEG 300, PEG 400, PEG 600, PEG 600, PEG 3350, PEG 400, poppyseed oil, propylene glycol, safflower oil, vegetable oil, etc.) chelating agents (such as calcium disodium EDTA, disodium EDTA, sodium EDTA, calcium versetamide Na, calteridol, DTPA), or other excipients.

In addition to solvent, oxygen, and/or oxygenating compositions, a liquid dosage form for IV, injection (e.g. intraocular injection, sub-retinal injection, intrathecal, directly into the heart), topical (e.g. to an eye), or oral administration to a mammal, including a human being, may contain a cyclodextrin, a cyclodextrin derivative, and/or a salt thereof, such as a naturally-occurring cyclodextrin (e.g., α, β, or γ-cyclodextrins) or a synthetic cyclodextrin. Examples include, but are not limited to, (2,3,6-tri-O-acetyl)-α-cyclodextrin, (2,3,6-tri-O-methyl)-Ε-cyclodextrin, (2,3,6-tri-O-octyl)-α-cyclodextrin, 6-bromo-6-deoxy-α-cyclodextrin, 6-iodo-6-deoxy-α-cyclodextrin, (6-O-tertbutyl-dimethylsilyl)-α-cyclodextrin, butyl-α-cyclodextrin, succinyl-α-cyclodextrin, (2-hydroxypropyl)-α-cyclodextrin, hydroxypropyl-β-cyclodextrin, 6-monodeoxy-6-monoamino-β-cyclodextrin, glucosyl-β-cyclodextrin, maltosyl-β-cyclodextrin, 6-O-α-D-glucosyl-β-cyclodextrin, 6-O-α-maltosyl-β-cyclodextrin, 6-azido-6-deoxy-β-cyclodextrin, (2,3-di-O-acetyl-6-O-sulfo)-β-cyclodextrin, methyl-β-cyclodextrin, dimethyl-β-cyclodextrin (DMBCD), trimethyl-β-cyclodextrin (TMBCD), (2,3-di-O-methyl-6-O-sulfo)-β-cyclodextrin, (2,6-di-O-methyl)-ß-cyclodextrin, (2,6-di-O-ethyl)-β-cyclodextrin, (2,3,6-tri-O-methyl)-β-cyclodextrin, (2,3,6-tri-O-acetyl)-β-cyclodextrin, -(2,3,6-tri-O-benzoyl)-β-cyclodextrin, (2,3,6-tri-O-ethyl)-β-cyclodextrin, 6-iodo-6-deoxy-β-cyclodextrin, 6-(dimethyl-tert-butylsilyl)-6-deoxy-β-cyclodextrin, 6-bromo-6-deoxy-β-cyclodextrin, monoacetyl-ß-cyclodextrin, diacetyl-β-cyclodextrin, triacetyl-β-cyclodextrin, (3-O-acetyl-2,6-di-O-methyl)-β-cyclodextrin, (6-O-maltosyl)-β-cyclodextrin, (6-O-sulfo)-β-cyclodextrin, (6-O-t-butyldimethylsilyl-2,3-di-O-acetyl)-β-cyclodextrin, succinyl-(2-hydroxypropyl)-β-cyclodextrin, (2,6-di-O-)ethyl-β-cyclodextrin, (2-carboxyethyl)-β-cyclodextrin (CMEBCD), hydroxyethyl-β-cyclodextrin (HEBCD), (2-hydroxypropyl)-β-cyclodextrin, (2-hydroxypropyl)-β-cyclodextrin (HPBCD), (3-hydroxypropyl)-β-cyclodextrin (3HPBCD), (2,3-hydroxypropyl)-β-cyclodextrin (DHPBCD), butyl-β-cyclodextrin, methyl-β-cyclodextrin, silyl((6-O-tert-butyldimethyl)-2,3,-di-O-acetyl)-β-cyclodextrin, succinyl-β-cyclodextrin, (2-hydroxyisobutyl)-β-cyclodextrin, randomly methylated-β-cyclodextrin, branched-β-cyclodextrin, sulfobutyl ether-β-cyclodextrin (e.g., SBEBCD, betadex, CAPTISOL®), carboxymethyl-γ-cyclodextrin, (2,3,6-tri-O-acetyl)-γ-cyclodextrin, (2,3,6-tri-O-methyl)-γ-cyclodextrin, (2,6-di-O-pentyl)-γ-cyclodextrin, 6-(dimethyl-tert-butylsilyl)-6-deoxy-γ-cyclodextrin, 6-bromo-6-deoxy-γ-cyclodextrin, 6-iodo-6-deoxy-γ-cyclodextrin, (6-O-t-butyldimethylsilyl)-γ-cyclodextrin, succinyl-γ-cyclodextrin, hydroxypropyl-γ-cyclodextrin (2-hydroxypropyl)-γ-cyclodextrin, acetyl-γ-cyclodextrin, butyl-γ-cyclodextrin, or combinations thereof.

A liquid dosage form comprising an oxygen-containing liquid, e.g. for IV, injection (e.g. intraocular injection, sub-retinal injection, etc.), topical (e.g. to an eye), or oral administration, to a mammal, including a human being, may have any suitable pH, such as about 2-12, about 2-4, about 4-6, about 6-8, about 8-10, about 10-12, about 6-7, about 7-8, about 8-9, about 6-6.5, about 6.5-7, about 7-7.5, about 7.5-8, about 8-8.5, about 8.5-9, about 7-7.2, about 7.2-7.4, about 7.4-7.6, about 7.6-7.8, about 7.8-8, or any pH in a range bounded by any of these values.

For many routes of administration, it may be helpful for the oxygen-containing liquid to be hypertonic or hyperosmolar, e.g. having a tonicity or an osmolarity greater than about 290 mOsm/L, such as about 290-600 mOsm/L, about 290-400 mOsm/L, about 400-500 mOsm/L, or about 500-600 mOsm/L; isotonic or isoosmolar, e.g. having a tonicity or an osmolarity near that of the body tissue to which it administered, such as about 290 mOsm/L, about 250-350 mOsm/L, about 250-320 mOsm/L, about 270-310 mOsm/L, or about 280-300 mOsm/L; or hypotonic or hypoosmolar, e.g. having tonicity or an osmolarity less than about 290 mOsm/L, such as about 150-290 mOsm/L, about 150-200 mOsm/L, about 200-290 mOsm/L, about 200-250 mOsm/L, or about 250-290 mOsm/L.

An oxygen-containing liquid may also potentially be delivered in microparticle delivery systems, nanoparticle delivery systems, nanoemulsion delivery systems, microemulsions delivery systems, microsomal delivery systems, liposomal delivery systems, or lysosomal delivery systems. For example, an oxygen containing liquid might be contained in a reverse micelle or inside a nanoparticle, nanoemulsion, microemulsion, microsome, liposome, or lysosome. In some embodiments, the oxygen containing liquid is contained within polymer nanoparticles (such as PLGA nanoparticles). In some embodiments, the delivery system is dispersed within an oxygen containing liquid for delivery. For example, nanoparticles, microparticles, liposomes, or lysosomes may contain an oxygen-containing liquid, and may further be dispersed within an oxygen-containing liquid.

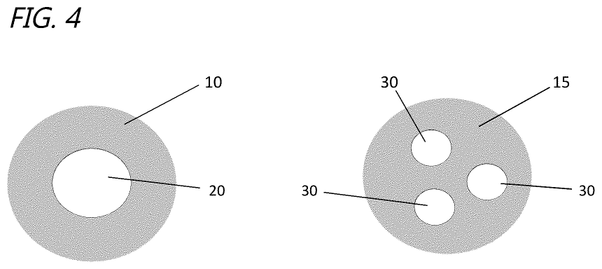

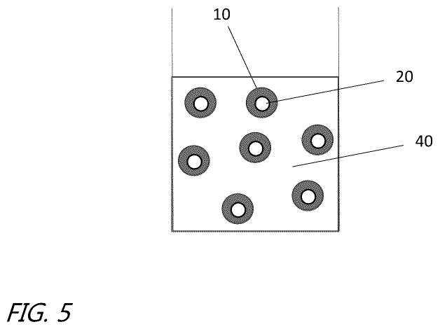

For example, as shown in FIG. 4, polymer 10 (e.g. PLGA) may form an exterior solid layer around oxygen-containing liquid center 20, or several smaller liquid nanodroplets or nanoparticles 30 may be encased in a larger solid polymer (e.g PLGA) host nanoparticle 15.

In some embodiments, a nanoparticle or a microparticle containing an oxygen-containing liquid may also be dispersed within an oxygen-containing liquid. For example, as shown in FIG. 5, polymer 10 (e.g. PLGA) may form an exterior solid layer around oxygen-containing liquid center 20, which may be dispersed within oxygen-containing liquid 40.

In addition to the above, it may be desirable for an orally administered liquid to contain a sweetening agent such as sucrose or saccharin; or a flavoring agent such as peppermint, methyl salicylate, or orange flavoring.

For creams, gels, ointments, etc. it may be desirable to include thickening agents, such as polyethylene glycol, polyacrylic acid, cetyl alcohol, stearyl alcohol, carnauba wax, stearic acid, hydroxyethylcellulose, guar gum, locust bean gum, xanthan gum, gelatin, silica, bentonite, magnesium aluminum stearate, etc.

A liquid dosage form comprising an oxygen-containing liquid might be part of a pharmaceutical product, which comprises the oxygen-containing liquid, an oxygen sensor, and a drug dispensing device. In some embodiments, the oxygen-containing liquid can only be dispensed if the oxygen-containing liquid has the desired oxygen pressure, such as an oxygen pressure described above.

While any suitable oxygen sensor may be used, a high performance microsensor available from Unisense is an example of a useful oxygen sensor.

Any suitable drug dispensing device may be used, such as a syringe or other form of injection device, a drop dispensing device.

Hypoxia, ischemia and reactive metabolites contributes to development and exacerbation of many disease states. The common denominator resulting in inhibition of tissue repair is tissue hypoxia.

Facilitating delivery of oxygen to tissues can result in adjunct and direct treatments in a wide variety of medical conditions.

Tissue hypoxia is low tissue oxygen level, usually related to impaired circulation. Tissue hypoxia, ischemia and reactive metabolites contribute to development and exacerbation of many disease states.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the Hypoxic Induction Factor (HIF) level of the tissue (e.g. eye tissue) having ischemia to be decreased by at least about 10%, at least about 20%, at least about 30%, at least about 40%, at least about 50%, at least about 60%, at least about 70%, at least about 80%, at least about 90%, or more as compared to the HIF level of the tissue (e.g. eye tissue) having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the HIF level of the tissue (e.g. eye tissue) having ischemia to be decreased so that it is within about 50%, within about 40%, within about 30%, within about 20%, within about 10%, within about 5%, within about 3%, or within about 1% of the HIF level of non-ischemic tissue (e.g. the contralateral eye).

In some embodiments, the reduction of the HIF level of the tissue may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, the reduction of the HIF level of the tissue may be continue for at least 1 day, at least 2 days, at least 3 days, at least 4 days, at least 5 days, at least 6 days, at least 7 days, at least 14 days, at least 21 days, or at least 28 days.

Administering or delivering an oxygen-containing liquid, such as a hyperbaric oxygen-containing liquid, to a mammal may be used to treat any type of ischemic condition, such as wounds, vasculopathies, malignant tumors, arthritis, atherosclerotic plaques, cancers, tumors, burns, inflammatory conditions, including inflammation of neural tissue (e.g. concussion), neuroinflammatory conditions, encephalitis, infection induced encephalitis, inflammation in the brain, infection in the brain, other inflammation induced by infection, or other inflammation related to inflammatory inducing conditions, respiratory infections or other conditions such as pneumonia, bronchitis, emphysema, asthma, etc.

In some embodiments, an oxygen containing liquid is used to oxygenate blood, such as human blood, without using a ventilator. For example, the oxygen containing liquid may be administered intravenously, or by another route, such as by oral administration. Oxygenating blood may be useful for treating any condition described herein.

An oxygen-containing liquid, such as a hyperbaric oxygen-containing liquid, may be used in conjunction with laser treatment or radiation treatment, e.g. to enhance the treatment effect or to assist in healing associated with the treatment.

Administering or delivering an oxygen-containing liquid, such as a hyperbaric oxygen-containing liquid, to a mammal may be used to treat a neurological condition such as Parkinson's disease.

In some embodiments, the ischemic condition is an ocular condition, such as diabetic retinopathy, macular degeneration, age related macular degeneration, dry age related macular degeneration, wet age related macular degeneration, macular edema, diabetic macular edema, glaucoma, sickle eye disease, ocular inflammation, hypertensive retinopathy, retinopathy of prematurity, ocular ischemic syndrome, branched retinal vein occlusion, branched retinal artery occlusion, central retinal vein occlusion, central retinal artery occlusion, retinal detachment, penetrating globe injury, traumatic optic neuropathy, optic neuritis, an inflammatory ocular condition, etc. In some embodiments, the ocular ischemic condition is diabetic retinopathy.

In some embodiments, the ocular ischemic condition is macular degeneration. In some embodiments, the ocular ischemic condition is diabetic macular edema. In some embodiments, the ocular ischemic condition is glaucoma. In some embodiments, the ocular ischemic condition is sickle cell eye disease. In some embodiments, the ocular ischemic condition is an ocular inflammation. In some embodiments, the condition is hypertensive retinopathy. In some embodiments, the condition is ocular ischemic syndrome. In some embodiments, the condition is retinal vein occlusion. In some embodiments, the condition is arterial occlusion, e.g. in the retina. In some embodiments, the condition is branched retinal vein occlusion. In some embodiments, the condition is branched retinal artery occlusion. In some embodiments, the condition is central retinal vein occlusion. In some embodiments, the condition is central retinal artery occlusion. In some embodiments, the condition is retinal detachment. In some embodiments, the condition is penetrating globe injury. In some embodiments, the condition is traumatic optic neuropathy. In some embodiments, the condition is optic neuritis. In some embodiments, the condition is an inflammatory ocular condition.

In some embodiments, the ischemic condition is one wherein the electrochemistry is altered, such as cardiovascular conditions, heart attack, stroke, neural ischemia, injury to the central nervous system, traumatic brain injury, spinal injury, acute and chronic traumatic encephalopathy, and immunocytotoxicity. Administering or delivering an oxygen-containing liquid, such as a hyperbaric oxygen-containing liquid, may also be useful to treat diseases or conditions related to, or caused by, sun damage or oxidation.

In some embodiments, an oxygen containing liquid may be used for the treatment of cancer. For example, the oxygen containing liquid may be administered in conjunction with a chemotherapy agent such as an alkylating agent, an antimetabolite, an anti-tumor antibiotic, a topoisomerase inhibitor, a mitotic inhibitor, etc. In some embodiments, co-administration of an oxygen containing with a chemotherapy drug can help to improve the activity of the chemotherapy drug or radiation therapy. For example, an oxygen containing liquid may increase oxygen within cancer and tumors to improve the effectiveness of radiation therapy and/or chemotherapy. An oxygen containing liquid may be used to reduce or halt tumor growth and/or growth rate. In some embodiments, a chemotherapy drug may be administered in an aqueous solution, e.g. intravenously or injected into the site of the cancer. An oxygen containing liquid may also have other therapeutic effects for the treatment of cancer.

Other conditions that may be treated with an oxygen containing liquid include anemia, migraine headaches, refectory osteomyelitis, a coronavirus infection (such as SARS-COV-2, which causes COVID-19), a viral infection, a bacterial infection, a fungal infection, etc., such as Enterobacter, Staphylococcus aureus including MRSA, Methicillin Resistant s. areus, Klebsiella pneumoniae, Actinomycetes, Pseudomonas, Enterococcus, Fungi, Mycobacterium, Enterococcus facium, S. Aureus, Klebsiella pneumonia, Actinobacter baumanii, Pseudomonas aeruginosa, Enterobacter, etc.

### Arenaviradae:

Tacaribe virus
Pichinde virus
Junin virus
Lymphocytic virus
Choriomeningitis virus

### Bunyaviridae:

Rift Valley Fever Virus
Punta Toro virus
LaCrosse virus
Maporal virus
Heartland virus
Severe Fever Thrombocytopenia Syndrome Virus
Oropouche virus

### Flaviviridae:

Dengue virus
West Nile Virus
Yellow fever virus
Japanese encephalitis virus
Powassan virus
Zika virus
Usutu virus

### Togaviridae:

Venezuelan equine encephalitis virus
Eastern equine encephalitis virus
Western equine encephalitis virus
Chikungunya virus
Mayaro virus

### Picornaviridae:

Poliovirus
Enterovirus-71
Enterovirus-68
Coxsackie virus B3

### Respiratory Viruses:

MERS coronavirus
Influenza A H1N1 virus
Respiratory syncytial virus

### Other Viruses:

Measles virus
Ebola virus
Lassa virus
Marburg virus
Nipah virus
Human Norovirus
Mouse Norovirus
HBV

### Herpesviridae:

Herpes simplex virus-1
Herpes simplex virus-2
Human cytomegalovirus
Murine cytomegalovirus
Guinea pig cytomegalovirus
Varicella-Zoster virus
Epstein-Barr virus
Human herpes virus-6b
Human herpes virus-8

### Papovaviridae:

BK virus
JC virus
Papillomavirus

### Other Viruses:

Adenovirus-5
Vaccinia virus
Cowpox virus

Oxygenation of a hypoxemic infected patient may be improved by administering an oxygen containing liquid to the patient. It is believed that an intravenous oxygen containing solution can help the body overcome hypoxemia and ameliorate organ damage. Intravenous oxygen delivery is a unique method of oxygenation. Intravenous oxygenation will allow faster recovery and help heal organs, avoiding the sometimes devastating consequences of hypoxemia from infections.

As disclosed herein, it has been discovered that an oxygen containing liquid can ameliorate consequences of retinal hypoxia. In vitro and in vivo studies have been conducted with some measure of success. It has been found that some oxygen containing liquids are safe across multiple cell lines. As described herein, oxygen containing liquids may protect cells in vitro and modulate the Vascular Endothelial Growth Factor (VEGF) response of cells exposed to a hypoxic environment (see Example 2 below). Additionally, as illustrated in Example 1 below, it has been demonstrated that some oxygen containing liquids can facilitate recovery of cellular function after induced ischemia.

Viral growth may be inhibited by high oxygen levels. Gene expression may be altered by oxidative conditions in infections. Hypoxia may directly or indirectly modulate viral response. Viruses and bacterial may target the hypoxic pathway and glycolytic metabolism. These mechanisms are complex and involve the Hypoxic Induction Factor (HIF) pathway and Vascular Endothelial Growth Factor (VEGF) regulation and how these relate to DNA or RNA viruses.

Some patients present with sepsis. A characteristic of sepsis is lactic acidosis. With an intravenous oxygen liquid lactic acidosis may potentially be overcome.

Infections in patients may be characterized using parameters measured by blood gas machines and other devices to measure oxygen (O₂), oxygen partial pressure (PO₂), carbon dioxide partial pressure (PCO₂), lactic acid, pH, electrolytes (such as Na⁺, K⁺, Ca²⁺, Mg²⁺, HCO₃⁻, CO₃²⁻, H₂PO³⁻, HPO₃²⁻, PO₃³⁻, etc.), and others. Blood gas machines and other devices may also be used to determine hypoxia and reactive oxygen species (ROS) in blood, such as superoxide, hydrogen peroxide, hydroxy radical, etc. Measured indexes of hypoxia, ROS and electrolyte variances in the blood can be used to characterize the disease severity, prognosis and staging. These blood tests can be used as markers in an algorithm which may affect treatment decisions.

Based upon the indices above, infection may potentially be treated and/or severity of the disease may potentially be ameliorated, and/or the progress of the disease may potentially be halted by intravenously administering an oxygen containing liquid. Reduction of organ damage and correction of hypoxemia and lactic acidosis may lessen the severity of the disease and/or halt virus propagation. This will afford the body a better opportunity to heal itself. This is especially true in conditions in which the level of hypoxia can indicate the severity of the disease.

Determining viral load may potentially be used as an indication of severity of infection. Based on viral load and indices of blood gas, blood measurements and hypoxia, infections may potentially be categorized into subcategories/subunits which will then provide guidance for treatment.

An infection, may be treated by administering an oxygen-containing liquid directly, e.g. by direct administration of the liquid or a form containing a liquid, such as a gel, cream, or a liquid phase dispersed in a solid (e.g. a biodegradable polymer). Treatment may also occur by indirect administration of an oxygen-containing liquid, such as by directly administering a solid or other non-liquid material that forms an oxygen containing liquid in situ, such as in a tissue or organ to be treated, in a gastrointestinal tract, or in another bodily fluid. In some embodiments, an oxygen-containing liquid is administered by intravenous injection, or by intravenous injection of a solid or other non-liquid material that forms an oxygen containing liquid in the patient's body after intravenous injection.

For treatment of viral infections, an oxygen containing liquid may be used alone, or in combination with a vaccine. For some vaccines, oxygenation may help to activate the vaccine or improve the efficacy of the vaccine.

In summary, in vivo and in vitro studies of oxygen containing liquids have shown that oxygen containing liquids can normalize increased VEGF levels in hypoxic environments, can be protective of cells exposed to a hypoxic environment, have excellent safety in vitro using 3 cell lines, and facilitate recovery from ischemic damage. Because of this, it is believed that administering an oxygen containing liquid can potentially reduce virus propagation and/or pathogenicity. It is also believed that administering an oxygen containing liquid can potentially supplement the human body's basic requirement of oxygen facilitating the body's own natural mechanisms to heal. Blood gas measurements, potentially with use of an algorithm, might be used to determine severity of disease before oxygen is required. Mobile blood gas machines are available.

An oxygen-containing liquid may also be administered to a mammal who is undergoing gene therapy, and may improve the outcome of the gene therapy. An oxygen-containing liquid may also be administered to a mammal in conjunction with treatment with stem cells, such as stem cells in the eye, e.g. retina, optic nerve, or other ocular structures.

An oxygen-containing liquid may also be administered to a mammal for improvement in blood oxygenation. This may be measured by transcutaneous oxygen measurement, pulse oximetry, or blood gas measurement.

An oxygen-containing liquid may also be administered to a mammal for improvement in vitreoretinal oxygenation, oxygenation of retina, oxygenation of subretina, or a combination thereof.

Improvement in many of the conditions described herein may be measured by optical coherent tomography (OCT), optical coherent tomography angiography, angiography, retinal oximetry, or some other imaging technique. Administering or delivering an oxygen-containing liquid, such as a hyperbaric oxygen-containing liquid, to a mammal may also be used to improve blood oxygen level in chronic diseases and to reduce the need for blood transfusions.

Administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, may result in an increase in ERG function of the ischemic tissue. For example, the scotopic b-wave response of an eye having ischemia may be about 0-5 mV, about 5-10 mV, about 10-15 mV, about 15-20 mV, about 20-50 mV, about 50-100 mV, or about 100-120 mV.

In some embodiments, administering or delivering an oxygen-containing liquid, such as a hyperbaric oxygen-containing liquid, to a mammal suffering from an ocular ischemic condition results in the scotopic b-wave response of the eye having ischemia to be increased by at least about 20 mV, at least about 30 mV, at least about 40 mV, at least about 50 mV, at least about 60 mV, at least about 70 mV, at least about 80 mV, at least about 90 mV, at least about 100 mV, or more, as compared to the scotopic b-wave response of the eye having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the scotopic b-wave response of the tissue (e.g. eye tissue) having ischemia to be increased by at least 2-fold, 3-fold, 4-fold, 5-fold, 6-fold, 7-fold, 8-fold, 9-fold, 10-fold, or more as compared to the scotopic b-wave response of the tissue (e.g. eye tissue) having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the scotopic b-wave response of the tissue (e.g. eye tissue) having ischemia to be increased so that it is within about 50%, within about 40%, within about 30%, within about 20%, within about 10%, within about 5%, within about 3%, or within about 1% of the scotopic b-wave response of normal or non-ischemic tissue (e.g. the contralateral eye).

In some embodiments, the improvement in ERG function may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the visual acuity of the mammal (e.g. human being) to be increased by about 10%, about 20%, about 30%, about 50%, about 70%, about 90%, or so that it is within about 50%, within about 40%, within about 30%, within about 20%, within about 10%, within about 5%, within about 3%, or within about 1% of the visual acuity of a normal eye (e.g.

the contralateral eye).

In some embodiments, the improvement in visual acuity may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the retinal thickness of the mammal (e.g. human being) to be decreased by about 10%, about 20%, about 30%, about 50%, about 70%, about 90%, or so that it is within about 50%, within about 40%, within about 30%, within about 20%, within about 10%, within about 5%, within about 3%, or within about 1% of the retinal thickness of a normal eye (e.g. the contralateral eye).

In some embodiments, the improvement in retinal thickness may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the neovascularization of the mammal (e.g. human being) to be reduced by about 10%, about 20%, about 30%, about 50%, about 70%, about 90%, or so that it is within about 50%, within about 40%, within about 30%, within about 20%, within about 10%, within about 5%, within about 3%, or within about 1% of the neovascularization of a normal eye (e.g. the contralateral eye).

In some embodiments, the improvement in neovascularization may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the Vascular Endothelial Growth Factor (VEGF) level of the tissue (e.g. eye tissue) having ischemia to be decreased by at least about 10%, at least about 20%, at least about 30%, at least about 40%, at least about 50%, at least about 60%, at least about 70%, at least about 80%, at least about 90%, or more as compared to the VEGF level of the tissue (e.g. eye tissue) having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the VEGF level of the tissue (e.g. eye tissue) having ischemia to be decreased so that it is within about 50%, within about 40%, within about 30%, within about 20%, within about 10%, within about 5%, within about 3%, or within about 1% of the VEGF level of normal or non-ischemic tissue (e.g. the contralateral eye).

In some embodiments, the reduction in the VEGF level of the tissue may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in an increase of the oxygen level (PO₂) or oxygen saturation (SO₂) by at least about 5%, at least about 10%, at least about 20%, at least about 30%, at least about 50%, or higher, as compared to the original PO₂ or SO₂. An increase from an original PO₂ of 50 to a level of 55 is an increase of 10%. In some embodiments, the SO₂ increases by at least about 5%, at least about 10%, at least about 20%, at least about 30%, at least about 50%, or higher, based upon a 100% saturation. An increase from 50% to 60% is an increase of 10% based upon a 100% saturation.

The following embodiments are specifically contemplated.

Embodiment D-1. A method of detecting infection, or detecting the progress of the disease, comprising: obtaining a blood level of a chemical species in a human being in need thereof.

Embodiment D-2. The method of Embodiment D-1, wherein the chemical species is a gas.

Embodiment D-3. The method of Embodiment D-2, wherein the gas is O₂.

Embodiment D-4. The method of Embodiment D-2 or D-3 wherein the gas is CO₂.

Embodiment D-5. The method of Embodiment D-1, D-2, D-3, or D-4, wherein the chemical species is lactic acid.

Embodiment D-6. The method of Embodiment D-1, D-2, D-3, D-4, or D-5, wherein the chemical species is H⁺, OH⁻, H₃O⁺, or a combination thereof, as determined by blood pH.

Embodiment D-7. The method of Embodiment D-1, D-2, D-3, D-4, D-5, or D-6, wherein the chemical species is an electrolyte.

Embodiment D-8. The method of Embodiment D-7, wherein the electrolyte is Na⁺.

Embodiment D-9. The method of Embodiment D-7 or D-8, wherein the electrolyte is K⁺.

Embodiment D-10. The method of Embodiment D-7, D-8, or D-9, wherein the electrolyte is Ca²⁺.

Embodiment D-11. The method of Embodiment D-7, D-8, D-9, or D-10, wherein the electrolyte is Mg²⁺.

Embodiment D-12. The method of Embodiment D-7, D-8, D-9, D-10, or D-11, wherein the electrolyte is HCO₃.

Embodiment D-13. The method of Embodiment D-7, D-8, D-9, D-10, D-11, or D-12, wherein the electrolyte is CO₃²⁻.

Embodiment D-14. The method of Embodiment D-7, D-8, D-9, D-10, D-11, D-12, or D-13, wherein the electrolyte is a phosphate species.

Embodiment D-15. The method of Embodiment D-7, D-8, D-9, D-10, D-11, D-12, D-13, or D-14, wherein the chemical species is a reactive oxygen species.

Embodiment D-16. The method of Embodiment D-15, wherein the reactive oxygen species comprises superoxide.

Embodiment D-17. The method of Embodiment D-15 or D-16, wherein the reactive oxygen species comprises hydrogen peroxide.

Embodiment D-18. The method of Embodiment D-15, D-16, or D-17, wherein the reactive oxygen species comprises hydroxy radical.

Embodiment D-19. Treatment of a virus by administering a therapy to a patient with a level of the chemical species of Embodiment D-1, D-2, D-3, D-4, D-5, D-6, D-7, D-8, D-9, D-10, D-11, D-12, D-13, D-14, D-15, D-16, D-17, or D-18 above a threshold value.

Embodiment D-20. Treatment of a virus by administering a therapy to a patient with a level of the chemical species of Embodiment D-1, D-2, D-3, D-4, D-5, D-6, D-7, D-8, D-9, D-10, D-11, D-12, D-13, D-14, D-15, D-16, D-17, or D-18 above a threshold value.

Embodiment D-21. The method of Embodiment D-19 or D-20, wherein the treatment comprises administering an oxygen-containing liquid to the patient.

Embodiment D-22. The method or treatment of Embodiment D-1, D-2, D-3, D-4, D-5, D-6, D-7, D-8, D-9, or D-10, further comprising determining a viral load in the human being. Embodiment D-23. A method of detecting infection by a virus, or detecting the progress of the disease, comprising: determining a viral load in a human being in need thereof.

Embodiment 1. A method of treating a mammal suffering from a condition related to ischemia, hypoxia, an alteration in electrochemistry, VEGF, HIF, or a reactive oxygen species, comprising delivering an oxygen-containing liquid to the mammal suffering from the condition, wherein the treatment results in a therapeutic effect on the condition.

Embodiment 2. The method of embodiment 1, wherein the condition is ocular and the oxygen-containing liquid is delivered to the eye of the mammal.

Embodiment 3. The method of embodiment 1 or 2, wherein the oxygen-containing liquid has an oxygen pressure that is higher than 140 mmHg.

Embodiment 4. The method of embodiment 1, 2, or 3, wherein the oxygen-containing liquid contains a compound that releases an oxygen gas.

Embodiment 5. The method of embodiment 1, 2, 3, or 4, wherein the oxygen-containing liquid has an osmolarity of about 250 mOsm/L to about 350 mOsm/L.

Embodiment 6. The method of embodiment 1, 2, 3, 4, or 5, wherein the oxygen-containing liquid comprises a metal oxide.

Embodiment 7. The method of embodiment 1, 2, 3, 4, 5, or 6, wherein the oxygen-containing liquid comprises a metal hydroxide.

Embodiment 8. The method of embodiment 1, 2, 3, 4, 5, 6, or 7, wherein the oxygen-containing liquid comprises a peroxide.

Embodiment 9. The method of embodiment 1, 2, 3, 4, 5, 6, or 8, wherein the oxygen-containing liquid is sterile.

Embodiment 10. The method of embodiment 1, 2, 3, 4, 5, 6, 7, 8, or 9, wherein the treatment results in an improvement of ERG function within 1 week of administering the oxygen-containing liquid to the eye of the mammal.

Embodiment 11. The method of embodiment 1, 2, 3, 4, 5, 6, 7, 8, 9, or 10, wherein the treatment results in a reduction in VEGF expression within 1 week of administering the oxygen-containing liquid to the eye of the mammal.

Embodiment 12. The method of embodiment 1, 2, 3, 4, 5, 6, 7, 8, 9, 10, or 11, wherein the condition is diabetic retinopathy.

Embodiment 13. The method of embodiment 1, 2, 3, 4, 5, 6, 7, 8, 9, 10, or 11, wherein the condition is macular degeneration.

Embodiment 14. The method of embodiment 1, 2, 3, 4, 5, 6, 7, 8, 9, 10, or 11, wherein the condition is diabetic macular edema.

Embodiment 15. The method of embodiment 1, 2, 3, 4, 5, 6, 7, 8, 9, 10, or 11, wherein the condition is sickle cell eye disease.

Embodiment 16. The method of embodiment 1, 2, 3, 4, 5, 6, 7, 8, 9, 10, or 11, wherein the condition is an ocular inflammation.

Embodiment 17. The method of embodiment 1, 2, 3, 4, 5, 6, 7, 8, 9, 10, or 11, wherein the condition is hypertensive retinopathy.

Embodiment 18. The method of embodiment 1, 2, 3, 4, 5, 6, 7, 8, 9, 10, or 11, wherein the condition is ocular ischemic syndrome.

Embodiment 19. The method of embodiment 1, 2, 3, 4, 5, 6, 7, 8, 9, 10, or 11, wherein the condition is branched retinal vein occlusion.

Embodiment 20. The method of embodiment 1, 2, 3, 4, 5, 6, 7, 8, 9, 10, or 11, wherein the condition is branched retinal artery occlusion.

Embodiment 21. The method of embodiment 1, 2, 3, 4, 5, 6, 7, 8, 9, 10, or 11, wherein the condition is central retinal vein occlusion.

Embodiment 22. The method of embodiment 1, 2, 3, 4, 5, 6, 7, 8, 9, 10, or 11, wherein the condition is central retinal artery occlusion.

Embodiment 23. The method of embodiment 1, 2, 3, 4, 5, 6, 7, 8, 9, 10, or 11, wherein the condition is retinal detachment.

Embodiment 24. The method of embodiment 1, 2, 3, 4, 5, 6, 7, 8, 9, 10, or 11, wherein the condition is penetrating globe injury.

Embodiment 25. The method of embodiment 1, 2, 3, 4, 5, 6, 7, 8, 9, 10, or 11, wherein the condition is traumatic optic neuropathy.

Embodiment 26. The method of embodiment 1, 2, 3, 4, 5, 6, 7, 8, 9, 10, or 11, wherein the condition is optic neuritis.

Embodiment 27. The method of embodiment 1, 2, 3, 4, 5, 6, 7, 8, 9, 10, or 11, wherein the condition is an inflammatory ocular condition.

Embodiment 28. The method of embodiment 1, 2, 3, 4, 5, 6, 7, 8, 9, 10, 11, 12, 13, 14, 15, 16, 17, 18, 19, 20, 21, 22, 23, 24, 25, 26, or 27, wherein the oxygen-containing liquid is injected into an eye of a human being.

Embodiment 29. The method of embodiment 1, 2, 3, 4, 5, 6, 7, 8, 9, 10, 11, 12, 13, 14, 15, 16, 17, 18, 19, 20, 21, 22, 23, 24, 25, 26, or 27, wherein the oxygen-containing liquid is topically administered to a human being.

Embodiment 30. The method of embodiment 1, 2, 3, 4, 5, 6, 7, 8, 9, 10, 11, 12, 13, 14, 15, 16, 17, 18, 19, 20, 21, 22, 23, 24, 25, 26, or 27, wherein the oxygen-containing liquid is orally administered to a human being.

### EXAMPLE 1

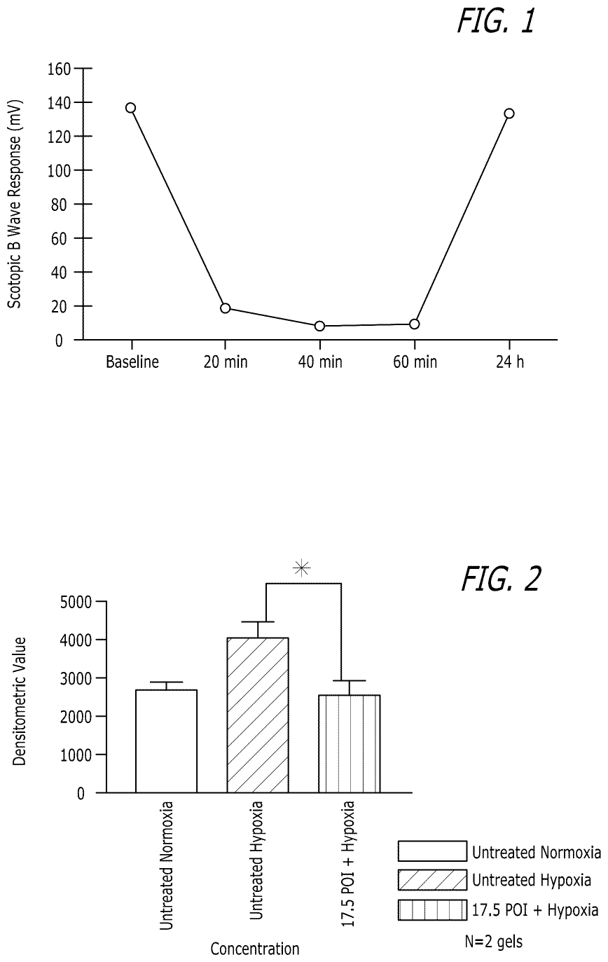

The effect of a hyperbaric oxygen solution in ischemic rabbit eyes was evaluated. Ischemia was induced in six rabbits as follows. A needle was connected to a saline bag, which was elevated to create pressure at the needle opening. The needle was placed into the rabbit eye and the intraocular pressure was allowed rise in the rabbit eyes for 90 minutes, which caused ischemia in the rabbit eyes. Rabbit 1 initially received no treatment, but then received an intraocular injection of the hyperbaric oxygen solution an hour after the needle attached to the saline bag was removed. Rabbits 2-3 were intraocularly injected with normal saline (with an oxygen pressure of 112.6 mmHg) 20 minutes after the needle attached to the saline bag was removed. Rabbits 4-6 were intraocularly injected with a hyperbaric oxygen solution (with an oxygen pressure of 175.2 mmHg) 20 minutes after the needle attached to the saline bag was removed. The results are depicted in Table 1 and FIG. 1.

TABLE 1

| | | Scotopic B Wave Response (mV) | | | | |
|---|---|---|---|---|---|---|
| | | | 20 min | 40 min | 60 min | 24 hr |
| | | | after | after | after | after |
| Rabbit | Treatment | Baseline | ischemia | ischemia | ischemia | ischemia |
| 1 | None | 136.9 | 18.52 | 7.8 | 8.85 | 133.4 |
| 2 | Saline | 155.4 | 16.65 | 12.03 | — | |
| 3 | Saline | 192.8 | 33.76 | 15.6 | 12.32 | |
| 4 | Hyperbaric | 136.1 | 11.46 | 22.17 | 25.8 | |
| | oxygen | | | | | |
| 5 | Hyperbaric | — | 19.43 | 28.4 | 48.6 | |
| | Oxygen | | | | | |
| 6 | Hyperbaric | — | 50.4 | 75.07 | 94.45 | |
| | Oxygen | | | | | |

### EXAMPLE 2

ARPE-19 cells were treated with a hyperbaric oxygen solution (oxygen pressure of 175.2 mmHg) and placed into a hypoxic chamber for 48 hours. Control cells were incubated in the hypoxic chamber without the hyperbaric oxygen solution. Phase contrast images show that the hypoxic ARPE-19 cells rounded up and showed unusual morphology compared to the hyperbaric oxygen treated hypoxic cells. There were 71 rounded cells per high power field in the control hypoxic cells versus 8 rounded cells per high power field in the hyperbaric oxygen solution treated hypoxic cells. It was concluded that hyperbaric oxygen solution appears to protect cells from the typical damage that results from exposure to hypoxia.

### EXAMPLE 3

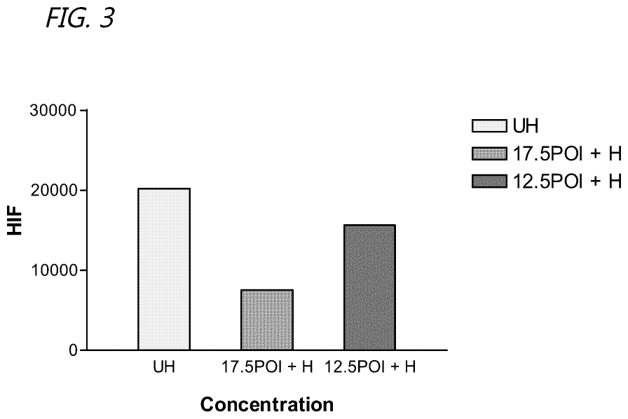

Retinal pigment epithelium cells were exposed to hypoxic conditions for 48 hours. Treatment with a hyperbaric oxygen solution (oxygen pressure of 175.2 mmHg) resulted in a statistically significant reduction in cellular levels of expressed vascular endothelial growth factor (VEGF) p<0.05 (FIG. 2) and HIF (FIG. 3).

As shown in FIG. 2, with 17.5% of an oxygenating ingredient added, the VEGF level of cells that had been exposed to hypoxic conditions (17.5POI+Hypoxia) was lower than the HIF level of cells that had been exposed to hypoxic conditions without treatment (Untreated Hypoxia), and was comparable to cells that had not been exposed to hypoxic conditions (Untreated Normoxia).

HIF level was analyzed by Western blot analyses. Proteins were extracted from the cell cultures and the protein concentrations measured with BCA protein Assay Reagent Kit (Pierce, Rockford, IL) according to the manufacturer's protocol.

As shown in FIG. 3, with 12.5% of an oxygenating ingredient added, the HIF level of cells that had been exposed to hypoxic conditions (12.5POI+H) was lower than the HIF level of cells that had been exposed to hypoxic conditions without treatment (UH). Furthermore, with 17.5% of an oxygenating ingredient added, the HIF level of cells that had been exposed to hypoxic conditions (17.5POI+H) was even lower.

These results indicate that treatment with a hyperbaric oxygen solution normalizes the VEGF and HIF levels of cells exposed to hypoxic conditions so that they are similar to the basal level of VEGF and HIF.

### Improved Blood Oxygenation

### EXAMPLE 4

The partial pressure of an oxygen containing solution was confirmed using a standardized blood gas machine. The PO₂ of the oxygen containing solution was 182 mmHg.

Blood plasma was taken from a single donor. Oxygen levels within blood plasma were measured on a standardized blood gas machine. Blood plasma was treated with a solution that contained oxygen levels measuring 182 mmHg (solution=182 mmHg O₂). Following treatment with the oxygen containing solution O₂ mmHg was immediately measured using a standardized blood gas machine. Measurements were taken at ten minutes post administration of the oxygenated solution.

TABLE 2
Blood Plasma Oxygen Levels

| | Pre Treatment | Post Treatment | Net gain in |
|---|---|---|---|
| | P0₂ mmHg | P0₂ mmHg | P0₂ mmHg |
| Sample 1 | 0₂ = 166 mmHg | 0₂ = 177 mmHg | 11 mmHg |
| Sample 2 | 0₂ = 170 mmHg | 0₂ = 185 mmHg | 15 mmHg |
| Sample 3 | 0₂ = 97.6 mmHg | 0₂ = 125 mmHg | 27.4 mmHg |

Notation: Mean time to observed improvement was within seconds. The largest change (27.4 mmHg was observed within 10 minutes of administration of solution).

Conclusion: Partial pressure of oxygen was increased in single donor plasma after treatment with a solution with measured increased partial pressure of oxygen.

### EXAMPLE 5

A sample of whole venous blood was taken from a single donor. Carbon dioxide level (pCO₂), oxygen level (PO₂), and oxygen saturation (SO₂) whole venous blood were measured on a standardized blood gas machine. The whole venous blood was treated with a solution that contained oxygen levels measuring 182 mmHg (solution=182 mmHg O₂). Following treatment with the oxygen containing solution, pCO₂, PO₂, SO₂ were immediately measured using a standardized blood gas machine. Measurements were taken at ten minutes and 20 minutes post administration of the oxygenated solution. Another treatment with the oxygen containing solution was added to the whole blood at 105 minutes after the first treatment and an additional measurement was taken. The results are depicted in Table 3.

TABLE 3
Whole Blood Oxygen Level

| | | | | | 105 min |
|---|---|---|---|---|---|
| | Whole | | | | post tx + |
| | blood | Immediate | 10 min | 20 min | (Additional |
| | baseline | Post Tx | post tx | post tx | tx) |
| pC0₂ | 56.4 | 51.7 | 52.7 | 42.5 | 32.6 |
| P0₂ | 46.8 | 48.2 | 49.4 | 52.6 | 59.1 |
| S0₂ | 79 | 80 | 80.7 | 84.3 | 90 |

Conclusions: After whole venous blood from a single donor was treated with a solution containing oxygen with PO₂ the following was observed:
1. Carbon Dioxide (pCO₂) decreased after treatment.
2. Oxygen level (PO₂) increased after treatment.
3. Saturation (SO₂) increased after treatment.

TABLE 5
Oxygen containing solution

| Time | Baseline | 2 min | 120 min |
|---|---|---|---|
| PCO2 | 51.1 | 48 | 50.1 |
| pH | 7.39 | 7.362 | 7.335 |
| PO2 | 23.9 | 24.2 | 25.5 |
| SaO2 | 41.6 | 43.8 | 44.9 |

| | | | | |
|---|---|---|---|---|
| Time | Baseline | 5 min | 30 min | 120 min |

Lyophilized

| | | | | |
|---|---|---|---|---|
| pH | 7.39 | 7.366 | 7.356 | 7.311 |
| PCO2 | 51.1 | 47 | 47.6 | 48.3 |
| PO2 | 23.9 | 26.6 | 27 | 27.9 |
| SaO2 | 41.6 | 49 | 48.8 | 51.3 |

Microparticles

| | | | | |
|---|---|---|---|---|
| pH | 7.39 | 7.337 | 7.332 | 7.317 |
| PCO2 | 51.1 | 52.1 | 52.7 | 54 |
| PO2 | 23.9 | 24.5 | 24.3 | 27.3 |
| SaO2 | 41.6 | 44 | 44.2 | 49.1 |

Nanoparticles

| | | | | |
|---|---|---|---|---|
| pH | 7.39 | 7.364 | 7.364 | 7.341 |
| PCO2 | 51.1 | 43.5 | 43.8 | 44.2 |
| PO2 | 23.9 | 33.4 | 33.8 | 37.5 |
| SaO2 | 41.6 | 65.1 | 65.7 | 67 |

1. A composition comprising nanoparticles or microparticles, wherein the nanoparticles or microparticles contain an oxygen containing liquid.

2. The composition of claim 1, wherein the nanoparticles or microparticles comprise poly (lactic-co-glycolic acid).

3. The composition of claim 1 or 2, for treating a condition related to ischemia or hypoxia.

4. A method of treating a condition related to ischemia or hypoxia, comprising administering a composition of claim 1 or 2 to a human being in need thereof, wherein the nanoparticles or microparticles contain an oxygen containing liquid.

5. The method of claim 4, wherein the oxygen containing liquid is directly administered to the central nervous system of the human being.

6. The method of claim 4, wherein the oxygen containing liquid is directly administered to the brain of the human being.

7. The method of claim 4, wherein the oxygen containing liquid is directly administered to an eye of the human being.

8. The method of claim 4, wherein the oxygen containing liquid is directly administered to the cardiopulmonary system of the human being.

9. The method of claim 4, wherein the oxygen containing liquid is directly administered to the heart of the human being.

10. The method of claim 4, wherein the oxygen containing liquid is directly administered to a lung of the human being.

11. The method of claim 4, wherein the oxygen containing liquid is directly administered to the liver of the human being.

12. The method of claim 4, wherein the oxygen containing liquid is directly administered to a kidney of the human being.

13. The method of claim 4, wherein the oxygen containing liquid is directly administered to the musculoskeletal system of the human being.

14. The method of claim 4, wherein the oxygen containing liquid is directly administered to a muscle of the human being.

15. The method of claim 4, wherein the oxygen containing liquid is directly administered to the endocrine system of the human being.

16. The method of claim 4, wherein the oxygen containing liquid is directly administered to the pancreas of the human being.

17. The method of claim 4, wherein the oxygen containing liquid is directly administered to the gastrointestinal system of the human being.

18. Use of a composition of claim 1 or 2, in the manufacture of a medicament for treating a condition related to ischemia or hypoxia.
